(19) United States
(12) Patent Application Publication
(10) Pub. No.: US 2026/0256391 A1
(43) Pub. Date: Sep. 3, 2026

(54) Biosensor for the in vivo monitoring of electrolytes

(71) Applicant: Proton Intelligence Inc., Gibsons (CA)

(72) Inventors: Esma Dervisevic, Melbourne (AU); Rita Roshni, Pakenham (AU); Citsabehsan Devendran, Balwyn North (AU); James Carthew, Melbourne (AU); Kaixuan Wang, Nottinghill (AU); Victor Javier Cadarso Busto, Mentone (AU); Jean-Baptiste Valsamis, Laguna Beach, CA (US); Sahan Ranamukhaarachchi, Narre Warren (AU)

(73) Assignee: Proton Intelligence Inc., Gibsons, BC (CA)

(21) Appl. No.: 19/489,963

(22) PCT Filed: Jun. 17, 2024
(86) PCT No.: PCT/US2024/034271

§ 371 Date: Dec. 4, 2025

Related U.S. Application Data
(60) Provisional application No. 63/523,060, filed on Jun. 24, 2023.

(51) Int. Cl. A61B 5/1473; A61B 5/145

(52) U.S. Cl. CPC A61B 5/1473; A61B 5/14546; A61B 2562/125

(57) ABSTRACT

In one embodiment, the present invention provides a layered composition for use with a biosensor for the in vivo monitoring of electrolytes. The layered composition includes a hydrophilic coating layer, and a hydrophobic ISM layer. In other embodiment, the present invention also provides biosensor containing such layered compositions and method of producing the layered compositions and biosensors described herein.

### CROSS-REFERENCE TO RELATED APPLICATIONS

The present application is a non-provisional utility application which claims the benefit of and priority to U.S. Prov. Pat. App. Ser. No. U.S. 63/523,060 (PROT.P-004-PV) filed on Jun. 24, 2023 and can be used alone or in combination with any of the compositions, devices and/or methods as described in: PCT/US2022/037198 (PROT.P-001-WO); PCT/US2022/052927 (PROT.P-002-WO); PCT/IB2023/061417(PROT.P-003-WO); U.S. Prov. Pat. App. Ser. No. U.S. 63/605,425 (PROT.P-005-PV); U.S. Prov. Pat. App. Ser. No. U.S. 63/556,008(PROT.P-006-PV); and U.S. Prov. Pat. App. Ser. No. U.S. 63/651,839(PROT.P-009-PV) which are all incorporated by reference for all purposes.

### BACKGROUND OF INVENTION

The selective detection of ions is usually achieved by means of a hydrophobic ion-selective membrane (ISM). The use of hydrophobic ISMs for in vivo applications, such as transdermal ion monitoring, requires the ISM to be biocompatible whilst maintaining a sensor performance that allows for an accurate and reliable analyte detection. A hydrophobic ISM is not suitable for in vivo use without any modifications, mainly because of two reasons; i) leaching of cytotoxic ISM components into the tissue, and ii) high biofouling caused by cells and proteins attached to the ISM surface. Moreover, leaching of ISM components and biofouling result in the loss of sensor lifetime in addition to impaired response accuracy, sensitivity, and reliability.

### SUMMARY OF INVENTION

The present invention solves problems of the art and provides specific coating layers for use with specific ISM layers which when combined can be employed in biosensors for continuous determination of analytes such as potassium and sodium. In a first embodiment, the present invention provides a layered composition for use with a biosensor for the in vivo monitoring of electrolytes where the layered composition includes a hydrophilic coating layer, and a hydrophobic ISM layer, preferably wherein these layers are in direct contact without the presence of an intervening layer.

In another embodiment, the present invention provides a layered biosensor for the in vivo monitoring of electrolytes, the biosensor including, in order a hydrophilic coating layer, a hydrophobic ISM layer, a transducer layer, and an electrode layer, preferably wherein each layer is in direct contact with its adjacent layer.

In a further embodiment, the present invention provides a method of forming a layered composition for use with a biosensor for the in vivo monitoring of electrolytes. The method includes the steps of: (a) forming or providing a hydrophobic ISM layer; (b) forming and applying a hydrophilic coating layer composition to the hydrophobic ISM layer formed or provided in step (a), optionally directed on top of and in contact with the ISM layer, thereby forming a layered composition for use with a biosensor for the in vivo monitoring of electrolytes.

In yet a further embodiment, the present invention provides a method of forming a biosensor for the in vivo monitoring of electrolytes. The method includes the steps of: (a) forming or providing an electrode layer; (b) forming or providing a transducer layer on the electrode layer formed or provided in step (a); (c) forming or providing a hydrophobic ISM layer on the transducer layer formed or provided in step (b); (d) applying a hydrophilic coating layer composition on the hydrophobic ISM layer formed or provided in step (c), and (e) applying the hydrophilic coating layer composition formed in step (b) to the hydrophobic ISM layer formed or provided in step (a), optionally wherein the layers are formed directly in contact with the adjacent layer formed in an earlier step.

### BRIEF DESCRIPTION OF DRAWING

FIG. 1 shows results from the example section where valinomycin levels were detected after 24 h, 48 h and 72 h with and without employment of an outer coating layer (OCL).

FIG. 2 shows results from the example section where cell viability data is provided demonstrating that OCL addition reduces silver ink toxicity through limiting the diffusion of components into external media.

FIG. 3 shows results from the example section where overall performance of the two OCLs is compared to the based ISM itself.

FIG. 4 shows results from the example section.

FIGS. 5, 6, and 7 shows aging data result from the example section of various ISM and coating layer configurations.

### DETAILED DESCRIPTION OF INVENTION

The present invention provides new compositions and methods and solves the problems of the prior art. In preferred embodiments, the present invention provides compositions for layer(s) of a biosensor for the in vivo monitoring of electrolytes, layered compositions useful as such biosensors, and methods of forming the same. The sensors are capable of the selective detection of ions. In some embodiments, to resolve the challenges in the art associated with the in vivo use of a hydrophobic ISM, the present invention provides a hydrophilic coating composition and/or layer for a biosensor, and in another embodiment a biosensor, composed of an electrode (e.g. a working electrode and a reference electrode) that includes at least one layer of electrically conductive electrode, an electrochemical transducer layer, a hydrophobic ISM, and a hydrophilic coating layer (e.g. preferably where the ISM covers only the working electrode). The sensor is preferably composed of three interfaces; i) tissue—ISM interface formed by the hydrophilic coating layer, ii) coating layer—transducer layer interface formed by the ISM, and iii) ISM—conductive electrode interface formed by the transducer layer.

### Definitions

### Electrically Conductive Electrode

A conductive material used to facilitate electron transfer, enabling electronic signal measurements. Such conductive materials can be formed from various conductive materials however in some preferred embodiments comprise gold.

### Transducer Layer

A layer that undergoes reversible chemical changes with respect to changing analyte concentrations resulting in conversion between chemical and electrical energy.

### Hydrophobic ISM

A membrane that contains selective analyte transport molecules such as ionophore selective to an ion, a lipophilic component that exchanges the target analyte with the analyte transport molecule, and a polymer matrix which may contain a plasticizer. An example of such an ISM is a potassium-selective membrane composed of potassium ionophore I, tetraphenyl borate lipophilic salt, and polyvinyl chloride as the membrane polymer plasticized with dioctyl sebacate. In certain embodiments, the hydrophobicity of the ISM is defined in comparison to the hydrophilicity of the coating layer. In these embodiments, the term hydrophobic as applied to properties of the ISM means that the ISM is more hydrophobic and/or less hydrophilic than the coating layer.

### Hydrophilic Coating

A coating layer applied on top of the hydrophobic ISM that enables the biosensor to be biocompatible, limits analyte diffusion and light penetration, and decreases the total biosensor impedance without compromising the analytical performance of the biosensor. In certain embodiments, the hydrophilicity of the coating is defined in comparison to the hydrophilicity of the coating layer. In these embodiments, the term hydrophilic as applied to properties of the coating layer means that the coating layer is more hydrophilic and/or less hydrophobic than the ISM.

### Embodiments

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) and/or feature(s) of any embodiment may be combined in any suitable manner with any other described embodiments.

### Numerical Values

Numerical values in the specification and claims of this application reflect average values. Furthermore, unless indicated to the contrary, the numerical values should be understood to include numerical values which are the same when reduced to the same number of significant figures and numerical values which differ from the stated value by less than the experimental error of conventional measurement technique of the type described in the present application to determine the value.

### A Preferred Coating Layer

### 1. Hydrophilic Coating Layer: Tissue—ISM Interface

a. Composition

Hydrophilic polymers (such as polyurethanes and/or others) are a class of polymers that exhibit a strong affinity for water, making them highly suitable for applications in the biomedical industry. These materials possess unique properties that allow them to absorb and retain significant amounts of water without losing their structural integrity. They usually present a hydrophilic surface, enabling the interaction with biological fluids and tissues, with reduced biofouling. These properties make them suitable for various biomedical applications, particularly in the development of medical devices and implants, drug delivery systems and tissue engineering. In the biomedical industry, hydrophilic polymers (such as polyurethanes) are used to create flexible and biocompatible materials that can mimic the properties of natural tissues.

The preferred coating polymer has hydrophilic (soft) and hydrophobic (hard) segments with the ability to absorb water, swell, and form a hydrogel when in an aqueous medium. Isocyanates are an important component in polyurethane synthesis. Either an aliphatic or an aromatic type of isocyanate can be used, however it has herein been found that the choice of isocyanate can affect the reactivity and properties of the final polymer. Aliphatic isocyanates are less reactive than the aromatic counterparts. Some of the isocyanates that could be employed during the synthesis of hydrophilic polyurethanes includes hexamethylene diisocyanate (HDI), isophorone diisocyanate (IPDI), toluene diisocyanate (TDI), xylene diisocyanate, methylenediphenyl diisocyanate (MDI), and alike.

Tuning the hydrophilicity of the synthesized polymer (e.g. polyurethane) depends on the choice of hydrophilic polyol. Some of the examples of polyols can include, but are not limited to polyethylene glycol, polyvinyl alcohol, polypropylene glycol, diethylene glycol, ethylene glycol or glycerol. In another example, the hydrophilicity can be increased by hydrophilic additives such as polyvinylpyrrolidone, polyethylene oxide, polyacrylic acid, and alike, and by the incorporation of cross-linking agents or chain extenders such as diamines, diacids, diols, and alike.

In another example, the surface hydrophilicity can be increased by plasma treatment or grafting hydrophilic functional groups such as amine, oxygen containing functional groups, and alike.

In another example, the polymer includes a photoinitiator to enable photosensitivity. Some examples of photoinitiators used in polyurethanes can be, but are not limited to, 1-hydroxycyclohexyl phenyl ketone, 2-Hydroxy-2-methyl-1-phenyl-propan-1-one, bis(2,4,6-trimethylbenzoyl)phenyl phosphine oxide or diphenyl(2,4,6-trimethylbenzoyl) phosphine oxide, combined with multifunctional acrylates and some additives such as plasticizers and stabilizers. This polymer can be obtained using a diisocyanate in combination with a polyol and a hydroxyl terminated acrylate, which is photosensitive. In another example, polyurethanes or polysaccharides such as polygelatins, modified with norbornene and thiol groups can be used to induce thiol-ene click reactions when exposed to UV or near-UV light (320 nm-500 nm). Subsequently, the regions of interest are irradiated with a defined wavelength of light via the use of an appropriate photomask, which initiates the polymerization process.

In another example, acridine containing polyurethanes can be used to enable photo-degradation of the polymer. The polymer can be obtained using acridine-diol monomers with polyethylene glycol (PEG) for the preparation of the hydrophilic polyurethane. This will allow the application of the coating polymer and subsequent selective removal of the polymer from undesired regions, enabled by exposure with UV or blue light (λ=320 nm-500 nm) via the use of an appropriate photomask.

In other preferred embodiments, the ISM and the coating layer both contain the same or similar base polymer (e.g. polyurethane). In the present embodiment, the base polymer of the ISM is tuned or selected to be more hydrophobic/less hydrophilic than the polymer of the coating layer. Similarly, in the present other other embodiments, the base polymer of the coating layer is tuned or selected to be less hydrophobic/more hydrophilic than the polymer of the ISM.

b. Obtaining a Hydrophilic Layer on ISM

The hydrophilic polymer can be dissolved in a volatile organic solvent such as cyclopentanone, tetrahydrofuran, or chloroform forming a concentrated solution that can be used to deposit the polymer by spin-coating, spray-coating, casting (e.g. drop-casting), ink-jet printing, electrodeposition, or screen printing on the ISM layer. The polymer can also be coated by dip-coating by immersing the electrode in the polymer solution. The thickness of the polymer should not deteriorate the sensor performance when limiting the penetrating light towards the ISM, and preventing the leaching of membrane components into the medium.

In another example, the coating layer is obtained from multiple layers of hydrophobic or hydrophilic polymers. ISM generally consists of hydrophobic materials due to their essential hydrophobic components such as ionophore, cation exchanger, and plasticizer. To overcome the adhesion issues that arise between hydrophobic surfaces and hydrophilic materials, a multistep membrane coating process is implemented such that the hydrophobicity of the coating polymer is gradually decreased from the sensor membrane towards the surface of the coating polymer.

In another example, the surface of the hydrophobic ISM is functionalized using for example organosilanes in order to improve the adhesion of the hydrophilic coating to the ISM.

c. Functionality
i. Biocompatibility

In one example, the hydrophilic coating layer can contain minimal surface roughness alongside comparable mechanical properties to the implanted tissue environment, helping to mimic that of the native extracellular matrix. These properties help to limit both protein adsorption and negate a mechanical mismatch, which can contribute to the onset of local inflammation and subsequent immune response. To achieve this, a preferred coating layer can incorporate either gelatine, gelatine methacrylate, polyurethanes, poly(ethylene glycol) or collagen based hydrogels, as their native biocompatibility, low immunogenicity and highly tunable mechanical properties would make them well suited for limiting the foreign body response.

As an alternative to stealthing strategies, a preferred coating polymer can consist of proteins to actively reduce the native immunogenic response. One example is the addition of a surface layer of osteopontin to reduce the foreign body reaction at the site of implantation. Further to this, through the direct incorporation of anti-inflammatory molecules/factors (such as corticosteroids, tyrosine kinase inhibitors, non-steroidal anti-inflammatory drugs (NSAIDs) or dexamethasone) into the hydrophilic coating polymer, the release can be modulated into the surrounding microenvironment, helping to directly inhibit both the immune response and biofouling. This control over molecule release can be achieved through targeted degradation of active molecule-polymer coating bonds over time, or alternatively through the incorporation of nanocomplexes into the polymer matrix, from which diffusion can be regulated through diffusion based mechanisms.

The hydrophilic coating layer facilitates the interaction of the native extracellular matrix with the sensor, enabling a signal stabilization within a very short time, such as one hour.

ii. Mass Transport Limitation

In one example, the hydrophilic coating layer limits the diffusion of the analyte towards the ISM to prevent signal saturation in a short time. In a logarithmic scale, this is particularly beneficial because the signal change decreases exponentially with respect to increasing analyte concentrations. The mass transport properties of water-soluble molecules can be decreased by decreasing the porosity of the hydrophilic coating polymer, which can be achieved by increasing the amount of chain extender incorporated during the synthesis of a hydrophilic polyurethane.

In another example, the hydrophilic coating polymer can contain proteins, amino acids such as lysine, or amines to facilitate cross-linking through aldehyde chemistry, or carbodiimide crosslinking chemistry, or photoinitiator to enable photo-induced crosslinking, where higher crosslinking results in smaller mass transport.

iii. Light Penetration Limitation

In one example, the biosensor contains a transducer layer such as poly(3-hexylthiophene) and alike, whose electron transfer properties are affected by the presence of light. To prevent non-selective changes in the biosensor response caused by light, an opaque hydrophilic coating layer is used, such as polysaccharite hydrogels cross-linked with N,N′-methylene bisacrylamide, glutaraldehyde, and alike, cryogels such as polyacrylamides which can contain a photoinitiator such as Irgacure 2959, and alike, and a crosslinker such as N,N′-methylene bisacrylamide, and alike.

In another example, the light penetration can be tuned by the mole amount of diisocyanate and chain extender components used during the synthesis.

iv. Prevention of Leaching of ISM Components

The leaching of cytotoxic ISM components can be slowed by increased porosity and crosslinking density achieved as illustrated in the examples given for limiting the mass transport towards the ISM.

In another example, a lipophilic component such as fatty acids, covalently bonded to the hydrophilic polymer can be incorporated such that the lipophilic toxic compounds leaching from the ISM can be entrapped in the coating layer.

v. Decrease the Impedance of the Biosensor

Electrochemical sensors are generating electrical signals depending on a large set of parameters such as ion concentrations, temperature, pressure, and alike. The electrical model involves different layers being modeled by resistors, capacitors, inductors, Warburg elements, voltage supply, current supply, and other electrical components.

Hydrophobic ISM can be modeled by high resistors (several hundreds of MOhm). Therefore, from an electrical standpoint, an ion-selective electrode presents a very high output impedance. This has been found problematic for the following reasons:
During Open Circuit Potentiometric (OCP) measurements, an electrical potential is generated between electrodes and is correlated to the concentration of the target analyte through the Nernst equation. The high output impedance makes the signal unstable to steady state condition and makes circuitry very sensitive to noise (electromagnetic interference such as 50 Hz or wireless communication, susceptibility to electrostatic charges such as the presence of a human body). Specific amplification layers, filtering and shielding are required to reduce/eliminate this type of electrical noise.
During Electrochemical Impedance Spectroscopy (EIS), the major source of the measured impedance is attributed to the resistance of the sensor membrane and not the desired impedance correlated to the change of target analyte concentration.
During other measurement techniques involving the measurement of an electric current or the flow of an electric current, the high impedance is considerably reducing the order of magnitude of such electrical current for a given electrical potential usually in the range of −15 V to +15 V.

In one example, the ISM contains a conductive polymer with low impedance. Different materials have different electrical conductivities as well as ion transport characteristics. Examples of low impedance conducting materials that can be incorporated into the ISM are, but not limited to, polyaniline and polythiophene. These polymers are known to have high electrical conductivity with low impedance. In another example, the impedance of the biosensor can be reduced by using a hydrophilic outer coating material.

d. Composition

An ISM contains a polymer matrix composed of hydrophobic polymers which include but are not limited to individually used, or a combination of, polyvinyl chloride, silicones, fluorosilicones, polyurethane, polyacrylates, and perfluoro polymers. The sensor membrane can also contain plasticizers including, but not limited to nitrophenyl octyl ether, dioctyl sebacate, dibutyl sebacate, and dioctyl phthalate, to increase the fluidity of the polymer matrix and membrane permeability to the target analyte. The sensor membrane contains an ion transport molecule such as ionophores which have a higher affinity to the target analyte compared to the remaining electrolytes, with the ability to transport; sodium such as ETH227 and alike, hydrogen such as tridodecylamine, ETH 1907, and alike, chloride such as ETH 9033 and alike, lithium such as ETH 2137 and alike, potassium such as valinomycin, BB15C5, and alike, calcium such as ETH 5234 and alike, magnesium such as K22B5, ETH 4030, and alike, among other ions through the ISM. The ISM also contains a lipophilic salt of tetraphenyl derivatives, N-(2,3,5,6,8,9,11,12-octahydro-16-nitro-1,4,7,10,13-benzopentaoxacyclopentadecin-15-yl)-, 2-dodecyl-2-methyl-1,3-propanediyl ester (BME 44), tridodecylmethylammonium salts, and alike. In one example, the ISM contains potassium ionophore II, potassium tetrakis(4-chlorophenyl) borate, dioctyl sebacate, and polyurethane when dissolved in a solvent such as tetrahydrofuran, cyclohexanone, and alike.

In another example, a photocurable ISM can be obtained by photopolymerization directly on the electrode surface where a solution containing polymer monomers such as methacrylate, acrylates, and alike, a photoinitiator such as 2,5-dimethyl-3′-methoxybenzophenone and alike, in addition to one or a combination of the above-mentioned plastisizers, lipophilic salts, and ionophores, when using a solvent such as dichloromethane, tetrahydrofuran, and alike is deposited on the electrode surface, and exposed to UV light.

e. Method of Deposition

A solution of ISM is obtained in a volatile solvent such as tetrahydrofuran, chloroform, cyclohexanone, dichloromethane and alike, which is used to obtain at least one layer of ISM on the transducer layer by drop-casting, spin-coating, spray-coating, or dip-coating.

In another example, a photocurable sensor membrane is selectively defined on the electrode by means of photolithography where the ISM solution is exposed to UV or near-UV light sources through a photomask, or to a laser light source with a micron-sized spot size, after which the non cross-linked areas are removed by dissolving them in an appropriate solvent that does not remove the cross-linked areas. The photocurable membrane can be obtained using azobisisobutyronitrile as a photoinitiator for polyvinyl chloride (PVC) based polymer membranes.

In another example, a non-toxic photolabile o-nitrobenzyl moiety can be introduced into a dextran backbone with an acrylate group to serve as a photodefinable masking layer. With the use of an appropriate photomask, desired membrane regions are irradiated with UV light to initiate the degradation and removal of the modified dextran layer. Subsequently, the ISM is applied on the substrate, followed by the dissolution of the modified dextran layer in water, leaving behind the ISM in desired regions only.

In another example, the ISM is deposited on the electrode area by means of, but not limited to spatial and volumetric controlled deposition, enabled by inkjet printing, ultra-low volume dispensers, and alike. Furthermore, the use of controlled film application via the use of a spin coater and doctor blade in conjunction with a stencil or screen mesh to define the regions of interest, after which the stencil is removed, leaving behind the sensor membrane in predefined locations only, akin to screen printing.

A preferred ISM can be formed from the compositions and methods as described in PCT/US2022/052927 (PROT. P-002-WO), which is incorporated herein by reference for all purposes.

f. Functionality

The ISM allows the selective analyte transport across the membrane towards the transducer layer. Its hydrophobicity creates a barrier between the aqueous medium and the transducer layer that disables non-selective signal generation.

### 2. Transducer Layer: ISM—Electrode Interface

a. Composition

The transducer layer can be obtained using self-assembled monolayers, metal and metal oxides nano and microstructures such as platinum nanostructures and molybdenum dioxide microparticles, and conductive polymers which are obtained by polymerizing one or a combination thereof when using materials including, but not limited to pyrrole, 1-hexyl-3,4-dimethylpyrrole, 3-octylthiophene, 3,4-ethylenedioxythiophene, 3-methylthiophene, aniline, indole, a-naphthylamine, o-anisidine, o-aminophenol, possibly in the presence of p-toluene sulfonate, polystyrene sulfonate, perfluorates, metallic complexes, and alike, and lipophilic ions such as tetrakis[3,5-bis(trifluoromethyl)phenyl borate and (4-chlorophenyl)borate, which can facilitate redox-activity, double layer capacitance, and ion-to-electron transduction.

b. Method of Deposition

The transducer layer can be deposited on the electrode surface by galvanostatic or voltammetric methods, where the conductive or semi-conductive polymer is attracted to the electrode surface with or without doping agents and counter ions by means of applied currents or potentials to the electrode. Examples include electrochemical polymerization of poly(3,4-ethylenedioxythiophene) doped with polystyrene sulfonate, polypyrrole doped with chloride ion, and alike.

In another example, the transducing material can be chemically attached to the electrode surface via covalent bonding, π-π interactions, electrostatic interactions, Lennard-Jones and Coulomb interactions.

In another example, a mixture of the transducing material with a volatile solvent such as tetrahydrofuran, ethanol, methanol, acetone, and alike can be obtained and deposited on the electrode surface by means of drop-casting, spin-coating, spray-coating and dip-coating.

In another example, the transducing material can be obtained by mixing a photosensitive material that allows the photodefinable patterning of the transducer layer on the electrode and substrate material.

In another preferred embodiment, a transducer layer can be formed on an electrode (e.g. gold electrode) surface using the compositions and methods as described in U.S. Prov. Pat. App. Ser. No. 63/425,658(PROT.P-003-PV) and the electrode can be formed from the compositions and methods as described in PCT/US2022/037198 (PROT.P-001-WO) which are incorporated in their entireties herein by reference for all purposes.

c. Properties

The transducer layer facilitates electron transduction in the presence of the target analyte, enabling a correlation between the analyte concentration with the recorded signal.

The transducer layer is preferably hydrophobic, improving the adhesion of the hydrophobic ISM and reducing/eliminating the accumulation of aqueous media at this interface, which is detrimental to the reliability of the acquired data.

### EXAMPLES

Without being bound by particular mechanism of action, the present invention is described further in the following section of examples which are illustrative of some of the embodiments herein described.

### Materials Description

Hydrophobic ISM 1: A polyurethane based ISM which contains a potassium ionophore 1 (valinomycin).
Hydrophobic ISM 2: A polyvinyl chloride based ISM which contains a potassium ionophore 1 (valinomycin).
Hydrophilic OCL 1: A polyurethane based hydrophilic polymer which has the water uptake ratio of ~15 %.
Hydrophilic OCL 2: A siloxane based hydrophilic polyurethane which has a water uptake ratio of ~20%.

### 1. Valinomycin Leaching

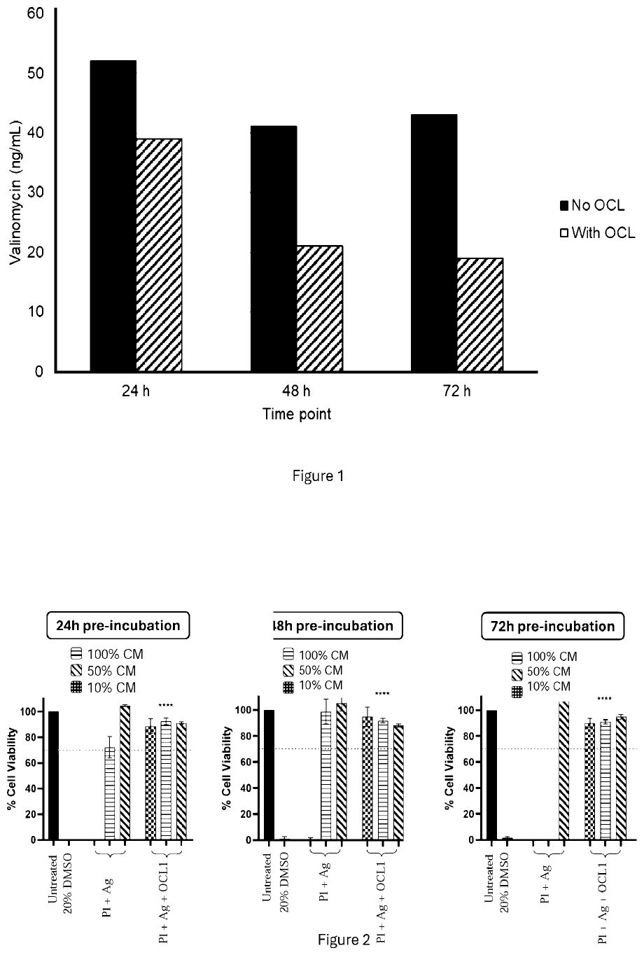

Two sensor configurations were prepared: 1. Hydrophobic PVC ISM w/o OCL, and 2. Hydrophobic PVC ISM with hydrophilic OCL 1. Ten sensors of each configuration are immersed in buffer solutions for different time periods: 24 hours, 48 hours, and 72 hours. This resulted in six test solutions. The extracts were analyzed for valinomycin levels using Liquid Chromatography Tandem Mass Spectroscopy. All batches of samples were analyzed using a calibration curve (R2≥0.99) in a range from 1 ng/ml to 1000 ng/ml. FIG. 1 shows valinomycin levels detected after 24 h, 48 h and 72 h with and without OCL.

### 2. OCL Inhibiting RR Leaching

1 μL droplets of Silver Ink were placed onto PI base substrates. Samples were then split into two groups, one with PI+Ag ink alone, and the second with the addition of 10 μL of OCL 1. Triplicate samples were then incubated in cell culture media at 37° C. for 24 h, 48 h and 72 h, following which, indirect cytotoxicity was assessed on L929 cells for 24 h using the CCK-8 cytotoxicity assay. FIG. 2 shows cell viability data demonstrating that OCL addition reduces silver ink toxicity through limiting the diffusion of components into external media.

### 3. ISM With OCL

Six configurations are tested in a homemade flow cell for 12 hours. The configurations are: Hydrophobic PVC ISM; Hydrophobic PVC ISM with OCL 1; Hydrophobic PVC ISM with OCL 2; Hydrophobic PU ISM; Hydrophobic PU ISM with OCL 1; Hydrophobic PU ISM with OCL 2. The KCI concentration in the flow cell is changed sequentially: 2 mM, 6 mM, 4 mM, and 8 mM, with each concentration maintained for 1 hour.

### Hydrophobic PVC ISM With OCL

Different combinations of PVC ISM with two different OCLs were constructed and evaluated for sensor response towards K+. A long term experiment was conducted in phosphate buffered saline to measure the potential when a specified amount of K+ was spiked into the electrochemical cells containing a working electrode (either made with a hydrophobic ISM with OCL 1 or OCL 2) and a reference electrode. The data was recorded by spiking 2 mM, 6 mM, 4 mM and 8 mM K+ in a series for 3 consecutive rounds over a 12 h time frame.

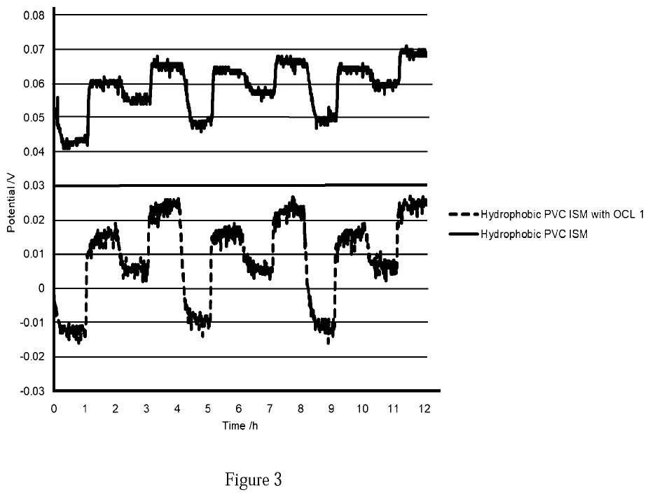

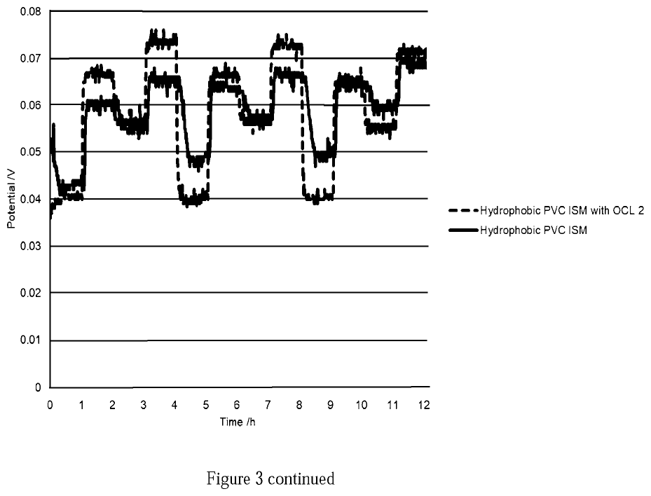

Performance: As shown in FIG. 3, overall, all the configurations respond to the K+ changes, however, the PVC ISM with OCL 1 and OCL 2 show superior performance compared to the PVC ISM counterpart. This performance could be attributed to the reduction in the leaching of the active component i.e. valinomycin from the ISM. Of the two OCLs, OCL 2 shows superior performance compared to OCL 1, attributed to its hydrophilic properties.

### Hydrophobic PU ISM With OCL

Different combinations of PU ISM with two different OCLs were constructed and evaluated for sensor response towards K+. A long term experiment was conducted in phosphate buffered saline to measure the potential when a specified amount of K+ was spiked into the electrochemical cells containing a working electrode (either made with a hydrophobic ISM with OCL 1 or OCL 2) and a reference electrode. The data was recorded by spiking 2 mM, 6 mM, 4 mM and 8 mM K+ in a series for 3 consecutive rounds over a 12 h time frame.

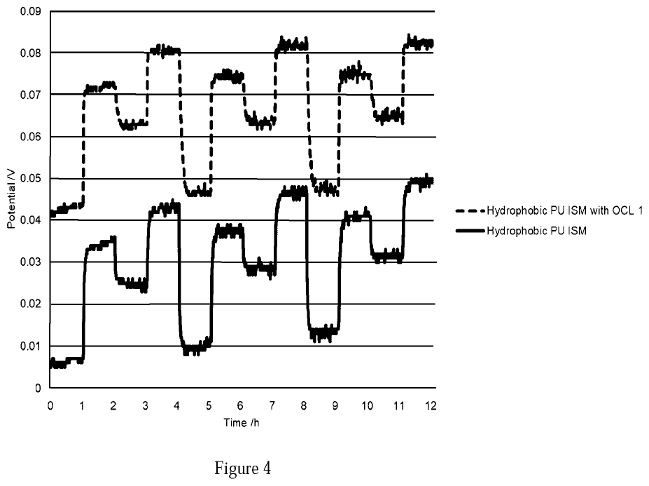

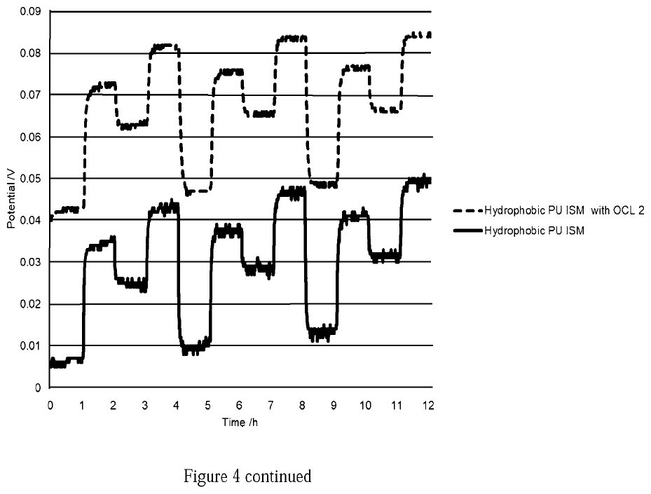

Performance: As shown in FIG. 4, in long-term flow cell test, considering the noise level, the hydrophobic PU ISM-based sensor showed the largest noise, the sensors with OCL 2 showed smallest noise. For the drift, the hydrophobic PU ISM-based sensor has positive drift, while the other two groups showed negligible drift.

### Life-Time Experiment up to 72 Hrs: PU ISM Only

Three configurations are tested: Hydrophobic PU ISM, Hydrophobic PU ISM with OCL1 and Hydrophobic PU ISM with OCL 2. They are evaluated under Open Circuit Potential (OCP) with K+ concentrations of 2 mM, 4 mM, 6 mM, and 8 mM on Day 1. Afterward, they are kept in a conditioning solution (2 mM KCl in PBS) for 72 hours, and the OCP test is repeated on Day 3.

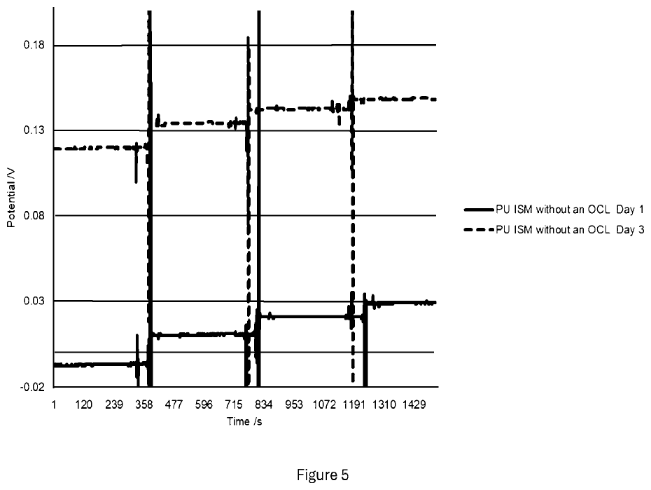

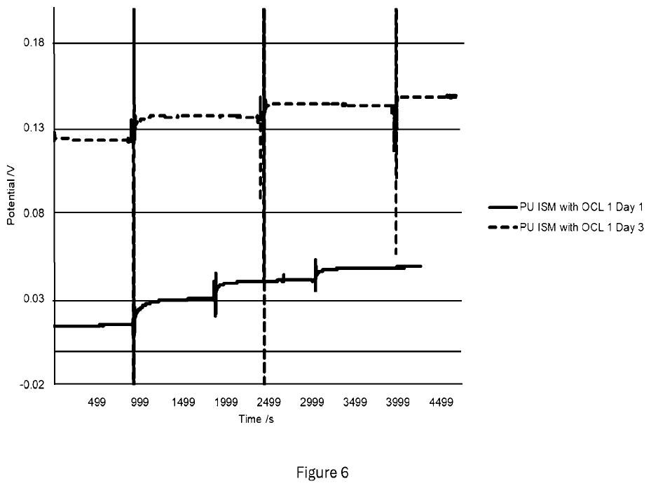

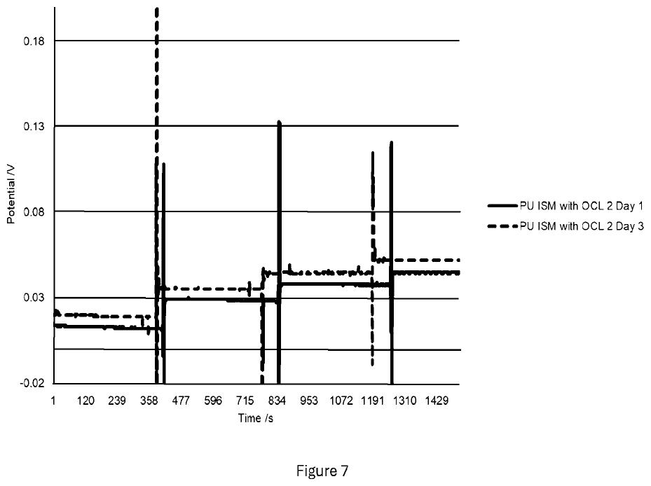

Performance: As show in FIGS. 5, 6, and 7, For hydrophobic PU ISM-based sensor, they have straight and fast response (<5 s), but they showed significant sensitivity drop after 3 days of conditioning (e.g. from 55 to 40 mV per decade); for hydrophobic PU ISM with OCL 1 based sensors, they have slow response (5-10 min) and they showed moderate sensitivity losing after 3 days of conditioning (e.g. from 55 to 50 m V per decade). Considering our sensitivity requirement is higher than 45 mV per decade, the performance of such sensors is acceptable; for hydrophobic PU ISM with OCL2 based sensors, they have straight and fast response (<5 s), and they didn't show sensitivity loss after 3 days of conditioning. FIG. 5 shows aging data for the hydrophobic PU ISM without an outer coating layer. FIG. 6 shows improved aging data for the same PU ISM however with OCL 1. FIG. 7 shows improved aging data for the same PU ISM however with OCL 2.

1. A layered composition for use with a biosensor for the in vivo monitoring of electrolytes, the layered composition comprising:
a hydrophilic coating layer, and
a hydrophobic ISM layer.

2. The layered composition of claim 1, wherein the hydrophilic coating layer comprises a polyurethane formed from an isocyanate and a polyol.

3. The layered composition of claim 2, wherein:
the isocyanate is an aliphatic isocyanate selected from the group consisting of: hexamethylene diisocyanate (HDI), isophorone diisocyanate (IPDI), toluene diisocyanate (TDI), xylene diisocyanate, methylenediphenyl diisocyanate (MDI), and alike; and
the polyol is a hydrophilic polyol selected from the group consisting of: polyethylene glycol, polyvinyl alcohol, polypropylene glycol, diethylene glycol, ethylene glycol, and glycerol; and
the hydrophilic coating layer and/or polyurethane optionally further comprises: a hydrophilic additive selected from the group consisting of polyvinylpyrrolidone, polyethylene oxide, polyacrylic acid, and alike; a cross-linking agents and/or chain extenders selected from the group consisting of diamines, diacids, diols, and alike; preparation via plasma treatment or grafting hydrophilic functional groups such as amine, oxygen containing functional groups, and alike; preparation with a photoinitiator.

4. The layered composition of claim 3, wherein the hydrophilicity of the polyurethane is tuned by selection of isocyanate, the polyol, or both the isocyanate and the polyol.

5. The layered composition of claim 1, further comprising a transducer layer and an electrode layer, wherein the electrode layer comprises a working and counter electrode, and wherein the transducer layer is in contact with the working electrode.

6. A layered biosensor for the in vivo monitoring of electrolytes, the biosensor comprising, in order:
a hydrophilic coating layer,
a hydrophobic ISM layer,
a transducer layer, and
an electrode layer,
optionally wherein each layer is in direct contact with its adjacent layer.

7. The biosensor of claim 6, wherein the hydrophilic coating layer comprises a polyurethane formed from an isocyanate and a polyol, wherein:
the isocyanate is an aliphatic isocyanate selected from the group consisting of: hexamethylene diisocyanate (HDI), isophorone diisocyanate (IPDI), toluene diisocyanate (TDI), xylene diisocyanate, methylenediphenyl diisocyanate (MDI), and alike; and
the polyol is a hydrophilic polyol selected from the group consisting of: polyethylene glycol, polyvinyl alcohol, polypropylene glycol, diethylene glycol, ethylene glycol, and glycerol; and
the hydrophilic coating layer and/or polyurethane optionally further comprises: a hydrophilic additive selected from the group consisting of polyvinylpyrrolidone, polyethylene oxide, polyacrylic acid, and alike; a cross-linking agents and/or chain extenders selected from the group consisting of diamines, diacids, diols, and alike; preparation via plasma treatment or grafting hydrophilic functional groups such as amine, oxygen containing functional groups, and alike; preparation with a photoinitiator.

8. The layered composition of claim 7, wherein the hydrophilicity of the polyurethane is tuned by selection of isocyanate, the polyol, or both the isocyanate and the polyol.

9. The biosensor of any of claim 7, wherein the electrode layer comprises a working and counter electrode, wherein the transducer layer is in contact with the working electrode.

10. A method of forming a layered composition for use with a biosensor for the in vivo monitoring of electrolytes, the method comprising the steps of:
(a) forming or providing a hydrophobic ISM layer;
(b) forming and applying a hydrophilic coating layer composition to the hydrophobic ISM layer formed or provided in step (a),
thereby forming a layered composition for use with a biosensor for the in vivo monitoring of electrolytes.

11. The method of claim 10, wherein the hydrophilic coating layer comprises a polyurethane formed from an isocyanate and a polyol, wherein:
the isocyanate is an aliphatic isocyanate selected from the group consisting of: hexamethylene diisocyanate (HDI), isophorone diisocyanate (IPDI), toluene diisocyanate (TDI), xylene diisocyanate, methylenediphenyl diisocyanate (MDI), and alike; and
the polyol is a hydrophilic polyol selected from the group consisting of: polyethylene glycol, polyvinyl alcohol, polypropylene glycol, diethylene glycol, ethylene glycol, and glycerol; and
the hydrophilic coating layer and/or polyurethane optionally further comprises: a hydrophilic additive selected from the group consisting of polyvinylpyrrolidone, polyethylene oxide, polyacrylic acid, and alike; a cross-linking agents and/or chain extenders selected from the group consisting of diamines, diacids, diols, and alike; preparation via plasma treatment or grafting hydrophilic functional groups such as amine, oxygen containing functional groups, and alike;
preparation with a photoinitiator.

12. The method of claim 10, wherein the hydrophilicity of the polyurethane is tuned by selection of isocyanate, the polyol, or both the isocyanate and the polyol.

13. The method of claim 10, where in step (b) the hydrophilic coater layer is applied to the hydrophobic ISM by a process selected from the group consisting of: spin-coating, spray-coating, casting (e.g. drop-casting), ink-jet printing, electrodeposition, and screen printing on the hydrophobic ISM layer.

14. The method of claim 10, wherein the hydrophobic ISM layer is disposed on a transducer layer and wherein the transducer layer is disposed on an electrode layer, and wherein the electrode layer comprises a working and counter electrode, wherein the transducer layer is in contact with the working electrode.

15. A method of forming a biosensor for the in vivo monitoring of electrolytes, the method comprising the steps of:
(a) forming or providing an electrode layer;
(b) forming or providing a transducer layer on the electrode layer formed or provided in step (a);
(c) forming or providing a hydrophobic ISM layer on the transducer layer formed or provided in step (b);
(d) applying a hydrophilic coating layer composition on the hydrophobic ISM layer formed or provided in step (c); and
(e) applying the hydrophilic coating layer composition formed in step (b) to the hydrophobic ISM layer formed or provided in step (a).

16. The method of claim 15, wherein the hydrophilic coating layer comprises a polyurethane formed from an isocyanate and a polyol, wherein:
the isocyanate is an aliphatic isocyanate selected from the group consisting of: hexamethylene diisocyanate (HDI), isophorone diisocyanate (IPDI), toluene diisocyanate (TDI), xylene diisocyanate, methylenediphenyl diisocyanate (MDI), and alike; and
the polyol is a hydrophilic polyol selected from the group consisting of: polyethylene glycol, polyvinyl alcohol, polypropylene glycol, diethylene glycol, ethylene glycol, and glycerol; and
the hydrophilic coating layer and/or polyurethane optionally further comprises: a hydrophilic additive selected from the group consisting of polyvinylpyrrolidone, polyethylene oxide, polyacrylic acid, and alike; a cross-linking agents and/or chain extenders selected from the group consisting of diamines, diacids, diols, and alike; preparation via plasma treatment or grafting hydrophilic functional groups such as amine, oxygen containing functional groups, and alike; preparation with a photoinitiator.

17. The method of claim 15, wherein the hydrophilicity of the polyurethane is tuned by selection of isocyanate, the polyol, or both the isocyanate and the polyol.

18. The method of any of claim 15, where in step (d) the hydrophilic coater layer is applied to the hydrophobic ISM by a process selected from the group consisting of: spin-coating, spray-coating, casting (e.g. drop-casting), ink-jet printing, electrodeposition, and screen printing on the hydrophobic ISM layer.

19. The method of any of claim 15, wherein the electrode layer comprises a working and counter electrode, wherein the transducer layer is in contact with the working electrode.
